(19) United States
(12) Patent Application Publication
(10) Pub. No.: US 2024/0180418 A1
(43) Pub. Date: Jun. 6, 2024

(54) DETERMINING OBJECTIVE MEASUREMENT DATA DURING A SUBJECTIVE REFRACTION

(71) Applicant: Rodenstock GmbH, Munich (DE)

(72) Inventors: Stephan Trumm, Muenchen (DE); Yohann Bénard, Muenchen (DE); Adam Muschielok, Muenchen (DE); Wolfgang Becken, Neuried (DE); Anne Seidemann, Muenchen (DE)

(21) Appl. No.: 18/549,301

(22) PCT Filed: Mar. 11, 2022
(86) PCT No.: PCT/EP2022/056293

§ 371 Date: Sep. 6, 2023

(30) Foreign Application Priority Data
Mar. 12, 2021 (DE) 102021202441.6

(51) Int. Cl. A61B 3/032; A61B 3/036; A61B 3/10; A61B 3/11

(52) U.S. Cl. CPC A61B 3/032; A61B 3/036; A61B 3/1015; A61B 3/112

(57) ABSTRACT

A method for determining objective measurement data of at least one eye of a user during a subjective refraction, including: acquiring subjective refraction data of the at least one eye in a first lighting state; acquiring and/or ascertaining pupillometric measurement data of the at least one eye in the first and a second lighting state different from the first lighting state; acquiring aberommetric measurement data of the at least one eye in the first and the second lighting state, or acquiring aberrometric measurement data of the at least one eye in the first or the second lighting state, and acquiring aberrometric measurement data of the at least one eye in the other one of the first and second lighting states taking into account the acquired and/or ascertained pupillometric measurement data of the at least one eye.

### TECHNICAL FIELD

The invention relates to a method, a device and a computer program for determining objective measurement data during a subjective refraction.

### BACKGROUND

In a subjective refraction, the refractive power of the optical correction is determined with which the eye(s) of a test person produce a sharp image of a visual object located, for example, in the distance. There are standardized methods for performing the subjective refraction. These methods are performed by a refractionist, such as an optician or an ophthalmologist. For this purpose, measuring devices such as trial glasses, test lenses and/or a phoropter are conventionally used. These measuring devices can be operated either manually or electrically by the refractionist.

In subjective refraction, the subjective visual impression of the test person is decisive for determining the required optical correction. In this process, the subject communicates with the refractionist by giving them feedback regarding a visual task they have been given.

To create highly optimized lenses, objective parameters of the eye(s) are used in addition to subjective refraction data. The objective parameters of the eye must be measured under at least two different conditions.

From DE 10 2011 120 973 A1, for example, a method is known of how the objective parameters required for this can be determined. Since the measuring devices for measuring the subjective refraction data are different from those for measuring the objective parameters, the measurement of the objective parameters is carried out separately from the measurement of the subjective refraction data.

### SUMMARY

The object of the invention is to provide an improved means of determining optical parameters of a glasses wearer, in which, in particular, well-matched measurement data can be acquired and/or in which the performance of the measurement is simplified.

This object is achieved by the subject matters of the independent claims. Preferred embodiments are the subject matters of the dependent claims.

One aspect relates to a method for determining objective measurement data of at least one eye of a user during a subjective refraction, comprising the steps of:
a) acquiring subjective refraction data of the at least one eye in a first lighting state;
b) acquiring and/or ascertaining pupillometric measurement data of the at least one eye in the first and a second lighting state different from the first lighting state; and
c) acquiring aberrometric measurement data of the at least one eye in the first and second lighting states;
   or
d) acquiring aberrometric measurement data of the at least one eye in the first or the second lighting state and ascertaining aberrometric measurement data of the at least one eye in the other one of the two lighting states taking into account the acquired and/or ascertained pupillometric measurement data of the at least one eye.

In the method, the individual method steps do not necessarily have to be carried out in the order listed above. This means that the individual method steps can take place either in the listed order, in a different order and/or also at least partially simultaneously. The method steps c) and d) are optional to one another, which means that the method is carried out either with the method steps a), b) and c) or with the method steps a), b) and d). The method can be used to record optical parameters of at least one eye, preferably both eyes, of a user. The optical parameters comprise both the subjective refraction data and, in addition thereto, objective parameters of the at least one eye, which can be determined from the aberrometric measurement data and/or the pupillometric measurement data. This optical parameter data can be used to design, create and/or manufacture highly optimized and/or customized lenses.

The subjective refraction data can be recorded and/or measured by means of a refraction unit. During the method step a), for example, the refraction unit can be used to manipulate curvatures of wavefronts and, if necessary, additionally a mean propagation direction and/or wavelength and/or intensity and/or a polarization state of a light emanating from a test image. The light manipulated in this way can be directed into at least one eye of the user. An optical correction can thus be defined to manipulate the light entering the eye. The optical correction can either be introduced in kind into the propagation path of the light or be simulated purely virtually by generating a corresponding wavefront.

When acquiring subjective refraction data, phoropters can be used as refraction units, in particular an automated phoropter, which can have refractive and/or diffractive elements such as lenses and/or grids as optical corrections. The subjective refraction can alternatively be performed using refraction glasses. In this case, refractive or diffractive elements can be adaptive as optical correction, i.e. designed, for example, as a deformable mirror and/or a deformable lens.

Alternatively or additionally, a light field display can be used to generate the test image(s) with simulated wavefront. The light field display can show one or more test images with associated correction simultaneously and/or consecutively as part of the determination of subjective refraction. Furthermore, the light field display can be used to realize and/or control at least one or both of the lighting states.

Methods for performing a subjective refraction, in which subjective refraction data of the user is recorded, are sufficiently known from the prior art.

In method step a), the subjective refraction data is acquired in the first lighting state.

When performing the method, a total of at least two different lighting states are set and used, namely the first lighting state and the second lighting state. Here, the first lighting state differs from the second lighting state in terms of brightness. Thus, one of the two lighting states can be used as a bright or brighter lighting state and the other lighting state can be used as a dark or darker lighting state. Therefore, the first lighting state is either brighter or darker than the second lighting state.

For example, the brighter lighting state can have a brightness of about 300 lux to about 1000 lux, which corresponds approximately to an average room brightness. An average brightness in a darkened room can be used as a darker state, for example a brightness of maximum 5 lux. For the creation of meaningful measurement data, the first and the second lighting state should preferably differ from each other by at least 50 lux, preferably by at least 100 lux, particularly preferably by at least 200 lux.

The subjective refraction is carried out during method step a) in the first lighting state, which can be designed as the brighter lighting state, for example. In any case, the first lighting state is bright enough to allow the user to perform a meaningful sight exercise as part of subjective refraction under its lighting conditions.

The subjective refraction and the subjective refraction data ascertained thereby relate to a determination of refraction data with the participation of the user as a test person. This refraction data obtains information regarding a sphere, additionally preferably also regarding a cylinder and/or an axis, of a perceived acceptable optical correction. The user's involvement is achieved by providing feedback on the quality of the visual impression during a sight exercise, for example, from predefined visual cues or other objects and/or scenes with upstream optical correction. Feedback can be given verbally, for example.

Pupillometric measurement data is acquired and/or ascertained in method step b). This can include measured pupillometric data, which comprises information on the size and/or shape of the pupil of the at least one eye. The information on the size and/or shape of the pupil may contain at least one piece of size information, for example a radius and/or a diameter of the pupil, in the case of an elliptical pupil e.g. a length of the major and/or minor axis. The information may also reflect the shape of the pupil in a more complex form, for example a pupil stroke, a pupil thickness and/or a shape different from a circular lens. In addition, the pupillometric data and/or measurement data may contain information on the position of the pupil, for example relative to a corneal vertex and/or to an optical axis of the eye. In particular, the pupillometric measurement data can be acquired and/or ascertained in exactly the same first lighting state in which the subjective refraction is also carried out in method step a). Therefore, the pupillometric measurement data acquired and/or ascertained in the first lighting state exactly matches the acquired subjective refraction data. Furthermore, the pupillometric measurement data is also acquired and/or ascertained in exactly the same lighting state(s) in which the aberrometric measurement data is also ascertained in method step c) or d). Therefore, the measurement data acquired or ascertained in this way matches well and can be correlated with each other.

The pupillometric measurement data can be measured using a pupil measuring unit. This pupil measuring unit can, for example, be designed as a camera with which at least one image recording of the pupil can be made. Alternatively, a Shack-Hartmann sensor can be used as the pupil measuring unit. In one embodiment, an aberrometry measuring unit used during method steps c) and/or d) may additionally be used as the pupil measuring unit. In this case, the aberrometry measuring unit acquires the size of patterns of illuminated points, taking into account any influences from optics located between the eye and the aberrometry measuring unit.

Since the first and second lighting states differ from each other in terms of their brightness, the pupillometric measurement data belonging to the first and second lighting states normally differs from each other as well.

In general, the term “acquiring” data may refer to a direct measurement of that data. The term “ascertaining” data can relate to an indirect calculation of data, in particular a calculation from other previously acquired and/or measured data, which may be related to the actual data to be ascertained. In method step b), for example, the pupillometric data can thus be partially (or completely) acquired and directly measured, and/or partially (or completely) calculated indirectly, i.e. ascertained from other data. Both terms, i.e. “ascertaining” and “acquiring”, can include saving, displaying and/or other providing of data.

In method step c), the aberrometric measurement data of the at least one eye is acquired in the first and second lighting states. This method step is a preferred embodiment, as the aberrometric measurement data of both the first and the second lighting state can be measured directly and thus acquired. It is worth noting here that the aberrometric measurement data is recorded in particular in exactly the same first lighting state in which the subjective refraction is also carried out in method step a). This is not easily possible with conventional devices, as the aberrometric measurement data is traditionally acquired separately from the subjective refraction, e.g. with other measuring devices. This normally changes the lighting conditions and the aberrometric measurement data is not available in exactly the same lighting state in which the subjective refraction is carried out. For example, looking through a phoropter to perform a subjective refraction changes the incidence of light on the user's eyes. If the phoropter is removed and a measuring device is used instead to acquire the aberrometric measurement data, the measurements are precisely not made at the same initial lighting state, but under at least somewhat different lighting conditions.

However, method step c) is carried out using exactly the same first lighting condition in which the subjective refraction is also carried out. As a result, the acquired optical data matches particularly well and can be used to create particularly high-quality eyeglass lenses.

The aberrometric measurement data thus comprises measured aberrometric data that contains information describing an aberration of the eye. This data contains information corresponding to at least the term of order defocus when represented with Zernike coefficients, preferably also data on astigmatism and/or higher order terms such as coma, trefoil error and/or spherical aberrations. In principle, it may be sufficient for the aberrometric measurement data to contain only 2nd order measurement data, e.g. data on defocus and/or astigmatism.

The aberrometric measurement data can be acquired by means of an aberrometric measuring unit, which can be designed as a Shack-Hartmann sensor, for example.

Although the implementation of method step c) is a preferred embodiment, the acquisition of the aberrometric measurement data is not always readily possible for both the first and the second lighting state. In order to be able to carry out the measurement in exactly the same initial lighting state in which the subjective refraction is also carried out, a special measuring device can be used. This measuring device can integrate both the refraction unit for acquiring the subjective refraction data and the aberrometry measuring unit for acquiring the aberrometric measurement data. In order to be able to change the lighting state sufficiently significantly in such a measuring device, a manipulation and/or deflection in the observation beam path by the refraction unit may be provided and/or necessary. This can be achieved, for example, by means of a manipulation device such as an aperture and/or a shutter in the observation beam path through the refraction unit. This manipulation device can be designed as an obscuration means and obstruct the acquisition of the aberrometric measurement data. For example, the manipulation device can obstruct and/or prevent wave propagation to the aberrometric measuring unit. Therefore, in practice it is not easy to achieve that the acquisition of the aberrometric measurement data according to method step c) can take place in both lighting states.

Therefore, the aberrometric measurement data is acquired according to the alternative method step d) in such a way that the aberrometric measurement data is acquired directly for at least one of these two lighting states. In particular, it can be the first lighting state, in which the observation beam path is open enough to perform the subjective refraction, which allows this direct measurement of the aberrometric measurement data.

With the help of the pupillometric measurement data already acquired for the two lighting states, the aberrometric measurement data acquired in this way (for one of the two lighting states) is now converted into the other of the two lighting states, i.e. for example from the first lighting state into the second or from the second lighting state into the first.

As a result, aberrometric measurement data for the first and second lighting states is provided after both method step c) and method step d). In method step c), the measurement data of both lighting states is measured directly and acquired in this way. In method step d), the measurement data for only one of the two lighting states is measured directly and thus acquired, while those for the other are ascertained by calculation with the aid of the pupillometric measurement data for the two lighting states.

The method can be performed either monocularly or preferably binocularly. Thus, sight exercises and/or visual objects in the distance can be used, in particular for subjective refraction, for example by means of a display such as a projection screen placed at a suitable distance. The method steps can be carried out alternatively or additionally for sight exercise and/or visual objects in the vicinity. Such sight exercises can be set using optotypes, visual and/or reading samples on different cards, and/or a controllable display. Such a display can be designed to be manually and/or automatically changeable.

The method allows simplified and/or accelerated acquisition of advanced optical parameters beyond the acquisition of subjective refraction data. Thus, the aberrometric measurement data and the pupillometric measurement data are additionally acquired as objective parameters. This objective measurement data is acquired in addition to the subjective refraction data for at least two different brightness conditions, preferably without the user having to switch between different measuring devices and without changing the brightness conditions in the measuring room.

According to this method, the aberrometric and pupillometric measurement data are acquired for the two different lighting states, before and/or during and/or after subjective refraction. Here, the subjective refraction takes place in exactly one of the two lighting states used, in particular in the brighter lighting state. In the brighter of the two lighting states, the average lighting density in the entrance pupil of the at least one eye of the user is higher than in the darker lighting state.

Acquiring aberrometric measurement data prior to subjective refraction allows the acquired aberrometric measurement data to be used as a target-guided starting point for subjective refraction.

Furthermore, a plausibility check of the subjective refraction can be carried out by means of the aberrometric measurement data, as well as a plausibility check of the aberrometric and pupillometric measurement data of the two lighting states against each other. The acquisition of the aberrometric measurement data can take place during the subjective refraction, wherein method steps c) and/or d) are carried out simultaneously or at least partially overlapping with method step a). The same applies to method step b), which can also be carried out simultaneously or at least partially overlapping with method step a).

In the method, a first aberrometric measurement may take place before and a second aberrometric measurement during subjective refraction. In this case, the previously performed aberrometric measurement can serve as the starting point of the subjective refraction, and the second aberrometric measurement can serve as a plausibility check of the subjective refraction.

An aberrometric measurement after the subjective refraction can be performed as an optical correction while holding the refraction values ascertained during the subjective refraction. In this way, the optical correction ascertained during subjective refraction can be checked.

During the subjective refraction, additional and/or further objective measurements may be taken to confirm the result of the subjective refraction. Thus, monocular and/or binocular optometric parameters can be ascertained and/or monocular and/or binocular sensitivity. The sensitivity of an eye is understood as the dependence of the visual acuity of this eye on a misrefraction. The misrefraction is a deviation from the ideal refraction for the eyes. In other words, sensitivity describes how much the visual acuity changes when an optical correction placed in front of the eye changes.

The objectively ascertained measurement data, i.e. the aberrometric measurement data and/or the pupillometric measurement data, can be used to calculate an optimized refraction. In this way, universal refraction data can be generated overall, which is matched to a high degree of accuracy due to the matching of the lighting states.

According to one embodiment, the method is carried out by means of a single measuring device into which a refraction unit for acquiring the subjective refraction data, a pupil measuring unit for acquiring the pupillometric measurement data and an aberrometry measuring unit for acquiring the aberrometric measurement data are integrated. A phoropter and/or refraction glasses can be used as a refraction unit. A wavefront sensor such as a Shack-Hartmann sensor can be used as the aberrometry measuring unit. For example, a camera, the aberrometry measuring unit and/or a separate wavefront sensor can be used as the pupil measuring unit. All of these units are integrated into the measuring device so that the method can be carried out using the same measuring device with exactly matching lighting states. This significantly improves the measurement data obtained and the optical parameters derived therefrom, as here the objective measurement data is precisely matched to the subjective refraction data. Advantageously, this also makes it possible to acquire subjective refraction data, aberrometric and/or pupillometric data for at least two brightness conditions without the need to change the test person over to another device or to change the brightness of the room. Further preferably, the measurements, in particular all of them, can be carried out without having to change and/or alter the position and/or orientation of the head of the test person, whereby advantageously the measurements can be carried out more quickly, more accurately and/or more comfortably for the test person. The measuring device may further have a control unit, e.g. with a processor. The control unit can be integrated into the measuring device or only connected to the measuring device. The control unit can be configured and/or used for evaluating and/or converting and/or processing the determined measurement data. For example, the control unit can be configured for image evaluation of image data acquired by the pupil measuring unit. In method step b), the control unit can be configured for ascertaining the aberrometric measurement data for the other lighting state. The control unit can generally contribute to and/or be configured for the acquisition, evaluation, conversion and/or processing of the data measured in method steps a), b), c) and/or d).

In an alternative embodiment, the brightness of the first or second lighting state is measured when performing the subjective refraction. Exactly this brightness is set and/or controlled when the pupillometric and/or aberrometric measurement data are acquired. This ensures that the subjective refraction and the acquisition of the pupillometric and/or aberrometric measurement data take place in the same lighting state.

In a further alternative embodiment, the brightness of the first or second lighting state is measured when acquiring the pupillometric and/or aberrometric measurement data. Exactly this brightness is set and/or controlled when performing the subjective refraction. This also ensures that the subjective refraction and the acquisition of the pupillometric and/or aberrometric measurement data take place in the same lighting state.

According to one embodiment, switching between the lighting states is performed without changing the ambient lighting condition. To change between the two lighting states, it is therefore not necessary to change the ambient light in the measuring room, but the change between the lighting states can be effected by the measuring device. Here, the lighting states relate in particular to the light hitting the user's eye(s). When performing the method, the user may, for example, look through an observation beam path of a refraction unit to perform subjective refraction. A switch between the lighting states can be effected by manipulating the light falling through the observation beam path.

According to one embodiment, a switch between the lighting states is effected by changing a brightness of a display unit. Additionally or alternatively, subjective refraction is performed along an observation beam path. Thus, a switch between the lighting states is effected by a manipulation of the observation beam path, wherein the manipulation is effected in particular by:
changing an aperture, and/or
operating a light source, and/or
operating a beam path interruption; and/or
operating a filter.

The observation beam path may pass through a refraction unit that is used for subjective refraction. The manipulation can be carried out by means of at least one manipulation device, which can be designed, for example, as the aperture, the light source, the beam path interruption, and/or the filter. For example, the darker lighting state can be achieved by shuttering the observation beam path through the refraction unit. In this case, the refraction unit with closed observation beam path can sufficiently darken the at least one eye. The aperture can be a blocking aperture, an opaque aperture, a pinhole aperture (for example with a variable opening) and/or a filter.

For example, in order to be able to precisely adjust the lighting brightness in the darker lighting state that has been darkened in this way, the light source can be used. The light source can, for example, be designed as an LED and/or a light bulb that illuminates the eye and/or can be switched on in a controlled manner. In doing so, the light source can use a non-blocked part of the observation beam path. This can be achieved, for example, by using the light source as a shutter of the observation beam path, and/or by reflecting the light of the light source through a (e.g. semi-)transparent mirror on the eye side in front of the shutter, and/or by a light source mounted on a housing of the refraction unit, which can, for example, shine obliquely into the at least one eye.

In this case, the subjective refraction in method step a) can also be carried out in the darker lighting state. Then the brighter lighting state can be produced by means of the switched-on light source.

The manipulation of the observation beam path by the refraction unit provides a particularly efficient way to realize the different lighting states.

Additionally or alternatively, the switch between the lighting states can be effected by changing a brightness of a display unit. The display unit can be the display unit on which sight exercises are displayed to the user in the context of subjective refraction. Since the user is looking at the display unit anyway, the brightness of the display unit thus also changes the brightness of the lighting state of the at least one eye in a simple way. The display unit can be designed as a display and/or as an appropriately illuminated display panel.

According to one embodiment, in method step d), the aberrometric measurement data of the at least one eye in the other of the two lighting states are ascertained by:
d1) representing the already acquired aberrometric measurement data in the first or second lighting state in a set of coefficients, in particular in Zernike coefficients, and scaling this set of coefficients by means of the acquired and/or ascertained pupillometric measurement data to the other of the two lighting states, and/or
d2) cutting out aberrometric data of a pupil shape according to the pupillometric measurement data acquired and/or ascertained for the other lighting state from the acquired aberrometric measurement data of a pupil shape according to the acquired and/or ascertained pupillometric measurement data belonging to the lighting state for which also the aberrometric measurement data have already been acquired, and/or
d3) extrapolating the already acquired aberrometric measurement data in the first or second lighting state to the pupil shape according to the acquired and/or ascertained pupillometric measurement data in the other lighting state.

Therefore, in this embodiment, method step c) is not carried out, but method step d) is carried out in at least one of the variants d1), d2), and/or d3). In one of the two lighting states, the aberrometric measurement data is measured directly and acquired as a measurement lighting state, so to speak. In the other of the two lighting states, the target lighting state, so to speak, direct measurement of the aberrometric measurement data may at least be hindered, for example by a blockage or restriction in the observation beam path. To acquire the aberrometric measurement data, it is not necessary to measure the aberrometric measurement data directly for both lighting states as in method step c). Rather, the aberrometric measurement data that has been directly measured in the measurement lighting state can be converted and/or transferred into the aberrometric measurement data of the target lighting state with the help of the pupillometric measurement data and thus ascertained. Generally, in method step d), the aberrometric measurement data of the target lighting state that has not yet been measured can be derived and/or calculated from the aberrometric measurement data of the measurement lighting state that has been measured directly. Here, the measurement lighting state is either the first lighting state or the second lighting state. Accordingly, the target lighting state is exactly the other of these two states, i.e. either the second lighting state or the first lighting state.

According to method step d1), the aberrometric measurement data already acquired at the measurement lighting state is represented in a set of coefficients. Zernike coefficients zₙ^{m}, for example, can be used for this purpose. These coefficients of the coefficient set, e.g. the Zernike coefficients zₙ^{m}, are scaled to the other lighting state, i.e. the target lighting state, for which no aberrometric measurement data is yet available, by means of the acquired pupillometric measurement data. For example, the Zernike coefficients zₙ^{m} of the target lighting state with pupil radius r₀ can be calculated from the Zernike coefficients zₙ^{m} of the measurement lighting state with pupil radius R₀ as follows:

$$\\ \begin{matrix}{𝓏}_{0}^{{}_{} 0} ( {r}_{0} ) = ⌊ {𝓏}_{0}^{{}_{} 0} ( {R}_{0} ) + ( {λ}^{2} - 1 ) ( \sqrt{3} {𝓏}_{2}^{{}_{} 0} ( {R}_{0} ) + \sqrt{5} ( 2 {λ}^{2} - 1 ) {𝓏}_{4}^{{}_{} 0} ( {R}_{0} ) + \sqrt{7} ( 5 {λ}^{4} - 5 {λ}^{2} - 1 ) {𝓏}_{6}^{{}_{} 0} ( {R}_{0} ) + … {}_{} ) ⌋ \\ {𝓏}_{1}^{{}_{} m} ( {r}_{0} ) = λ [ {𝓏}_{1}^{{}_{} m} ( {R}_{0} ) + ( {λ}^{2} - 1 ) ( 2 \sqrt{2} {𝓏}_{3}^{{}_{} m} ( {R}_{0} ) + \sqrt{3} ( 5 {λ}^{2} - 3 ) {𝓏}_{5}^{{}_{} m} ( {R}_{0} ) + 4 ( 7 {λ}^{4} - 8 {λ}^{2} + 2 ) {𝓏}_{7}^{{}_{} m} ( {R}_{0} ) + … {}_{} ) ] , m = - 1 , 1 \\ {𝓏}_{2}^{{}_{} m} ( {r}_{0} ) = {λ}^{2} [ {𝓏}_{2}^{{}_{} m} ( {R}_{0} ) + ( {λ}^{2} - 1 ) ( \sqrt{15} {𝓏}_{4}^{{}_{} m} ( {R}_{0} ) + \sqrt{21} ( 3 {λ}^{2} - 2 ) {𝓏}_{6}^{{}_{} m} ( {R}_{0} ) + … {}_{} ) ] , m = 0 , \pm 2 \\ {𝓏}_{3}^{{}_{} m} ( {r}_{0} ) = {λ}^{3} [ {𝓏}_{3}^{{}_{} m} ( {R}_{0} ) + ( {λ}^{2} - 1 ) ( 2 \sqrt{6} {𝓏}_{5}^{{}_{} m} ( {R}_{0} ) + 2 \sqrt{2} ( 7 {λ}^{2} - 5 ) {𝓏}_{7}^{{}_{} m} ( {R}_{0} ) + … {}_{} ) ] , m = \pm 1 , \pm 3 \\ {𝓏}_{4}^{{}_{} m} ( {r}_{0} ) = {λ}^{4} [ {𝓏}_{4}^{{}_{} m} ( {R}_{0} ) + \sqrt{35} ( {λ}^{2} - 1 ) {𝓏}_{6}^{{}_{} m} ( {R}_{0} ) + … {}_{} ] , m = 0 , \pm 2 , \pm 4 \\ {𝓏}_{5}^{{}_{} m} ( {r}_{0} ) = {λ}^{5} [ {𝓏}_{5}^{{}_{} m} ( {R}_{0} ) + 4 \sqrt{3} ( {λ}^{2} - 1 ) {𝓏}_{7}^{{}_{} m} ( {R}_{0} ) + … {}_{} ] , m = \pm 1 , \pm 3 , \pm 5 \\ {𝓏}_{6}^{{}_{} m} ( {r}_{0} ) = {λ}^{6} [ {𝓏}_{6}^{{}_{} m} ( {R}_{0} ) + … {}_{} ] , m = 0 , \pm 2 , \pm 4 , \pm 6 \\ {𝓏}_{7}^{{}_{} m} ( {r}_{0} ) = {λ}^{7} [ {𝓏}_{7}^{{}_{} m} ( {R}_{0} ) + … {}_{} ] , m = \pm 1 , \pm 3 , \pm 5 , \pm 7 \\ ⋮\end{matrix}$$

Here λ=r₀/R₀ is the ratio of the two pupil radii and zₙ^{m}(r) the Zernike coefficient of the radial order n and the azimuthal order m at the pupil radius r. This conversion is based on the assumption that the wavefront can also be represented sufficiently well by Zernike coefficients for the larger of the two radii (for example R₀). For practical reasons, wavefronts are preferred here whose Zernike representation already reproduces the wavefront well enough at a low order (e.g. 4 or 6). This is the case if no too high gradients are contained in the wavefront, which can normally only be well represented by very high orders.

In addition to the change of the pupil radius, the change of the pupil center and/or a rotation of the eye and thus also of the wavefront can be taken into account. Furthermore, the aberrometric measurement data of deformed pupils, in particular elliptical pupils, can be converted into each other.

Alternatively or additionally, a conversion can be performed according to step d2) by cropping the aberrometric data for a smaller pupil shape from the aberrometric measurement data for a larger pupil shape. This can be done in particular if the pupil is larger in the measurement lighting state than in the target lighting state, i.e. the lighting state for which the aberrometric measurement data must first be derived. To determine the aberrometric data of a smaller pupil, i.e. for example in the brighter lighting state, from the aberrometric measurement data of the larger pupil, i.e. for example in the darker lighting state, the measurement points, for example the measurement points of the Shack-Hartmann sensor, and/or derived quantities such as slope, arrow height and/or curvature of the wavefront can be cropped from the aberrometric measurement data with the larger pupil. Here, the aberrometric measurement data of the target lighting state lies within the aberrometric measurement data of the measurement lighting state. The desired aberrometric measurement data of the target lighting state can then be used as measurement points (for example of the Shack-Hartmann sensor) and/or derived quantities such as slope, arrow height and/or curvature of the wavefront of the smaller pupil in the brighter lighting state. This method according to method step d2) may be preferred exactly when the pupil in the target lighting state is smaller than the pupil in the measurement lighting state. This is advantageous because the actual wavefront of the smaller pupil is reconstructed and no assumptions regarding behavior have to be made.

According to method step d3), the already acquired aberrometric measurement data in the measurement lighting state is extrapolated to the pupil shape according to the acquired and/or ascertained pupillometric measurement data in the target lighting state. In order to infer from the aberrometric measurement data of one pupil in the measurement lighting state to the aberrometric data of the other pupil in the target lighting state, models of a behavior of the aberrations can be used that go beyond a mere scaling as used in method step d1). Thus, it can be assumed that the spherical aberration increases with the radius of the pupil. Within the framework of a model, this assumption can either be derived from nature, for example from models of the structure of the eye and its components, or empirically created, for example from measurements on eyes. This extrapolation can provide more accurate results than simply scaling the Zernike coefficients according to step d1). This also makes the ascertained aberrometric measurement data more accurate in the target lighting state.

According to one embodiment, in method step b)
b1) the pupillometric measurement data of the at least one eye (1) in at least one of the two lighting states directly measured by means of a pupillometric measurement of the pupil of the at least one eye (1) fully adapted to this lighting state performed during this lighting state; and/or
b2) the pupillometric measurement data of the at least one eye (1) indirectly ascertained in at least one of the two lighting states by means of a pupillometric measurement performed during this lighting state of the pupil of the at least one eye (1) which is currently adapting to this lighting state, namely
   after a switchover from another lighting state to this lighting state, and
   before the pupil has fully adapted to this lighting state, and
   by means of a conversion of this pupillometric measurement data measured during the adaptation to an adapted target state of the pupil in this lighting state.

According to an alternative or additional embodiment, in method step b)
b3) the pupillometric measurement data of the at least one eye (1) directly measured in at least one of the two lighting states by means of a pupillometric measurement of the pupil of the at least one eye (1) still fully adapted to this one of the two lighting states performed during another lighting state, and namely immediately after a switchover from this lighting state to the other lighting state and before the pupil starts adapting to the other lighting state; and/or
b4) the pupillometric measurement data of the at least one eye (1) indirectly ascertained in at least one of the two lighting states by means of a pupillometric measurement performed during another lighting state of the pupil of the at least one eye (1) which is currently adapting to this other lighting state, namely
   after a switchover from the lighting state for which the pupillometric measurement data are to be ascertained to the other lighting state, and
   while the pupil is still adapting to this other lighting state, and
   by means of a conversion of this pupillometric measurement data measured during the adaptation to an adapted initial state of the pupil in this original lighting state.

The method steps b1), b2), b3) and b4) listed above each show embodiments of how the pupillometric measurement data for the two lighting states can be determined in method step b). These method steps b1), b2), b3) and b4) can be combined with each other as desired. For example, the pupillometric measurement data can be measured for one of the two lighting states according to method step b1) and for the other lighting state according to one of the method steps b2), b3) or b4). Likewise, the pupillometric measurement data for the two lighting states can be determined using the same method, e.g. both using method step b1).

In the simplest case, the pupillometric measurement data can be measured directly with the pupil fully adapted for at least one of the two lighting states, in particular both the first and the second lighting state. This is done according to method step b1) and can be a preferred, particularly simple and/or accurate embodiment.

However, just as with the acquisition of aberrometric measurement data, direct measurement of pupillometric measurement data in at least one of the two lighting states can be difficult due to the geometry of the measuring device used and/or due to long adaptation times. Therefore, it may be the case that the fully adapted pupil cannot be measured directly and at rest for all two lighting states. In this case, a direct measurement of the adapted pupil can, for example, only be carried out for one of the lighting states, in particular during the first lighting state, during which the subjective refraction also takes place.

The pupillometric measurement data for at least one of the two lighting states, i.e. for the desired lighting state, can and/or should possibly not be measured directly and/or not necessarily with the pupil adapted or fully adapted. However, this pupillometric measurement data can still be sensibly acquired and/or ascertained according to at least one of the method steps b2), b3) or b4).

When switching between different lighting states, care should be taken to ensure that the subjective refraction and/or other measurements are not normally performed until the pupil has adjusted to the respective lighting condition, i.e. when the pupil has adapted.

However, the pupillometric measurement data of the desired lighting state can also be measured on a pupil that has not yet been adapted, i.e. either on a pupil that is still completely de-adjusted, as in method step b3), or on a pupil that is just adapting, as in one of the method steps b2) or b4).

Method steps b1) and b3) have in common, for example, that the pupillometric measurement data is measured directly. According to method step b1), however, measurements are also taken in exactly the same lighting state for which the pupillometric measurement data is also acquired, while according to method step b3) measurements are measured in a different lighting state.

Method steps b2) and b4) have in common, for example, that the pupillometric measurement data is not measured directly while the pupil to be measured is in an adaptation process. A pupil adaptation model can be used for this purpose. In method step b2), the pupil adaptation is progressively calculated into the future by means of the model, while in method step b4), the pupil adaptation is calculated back to an original state by means of the model.

The method steps b1) and b2) have in common, for example, that the pupillometric measurement data is measured exactly in the lighting state for which it is to be ascertained or acquired. In method step b1) it is measured directly, while in method step b2) it is calculated progressively into the future by means of a model for pupil adaptation.

Method steps b3) and b4) have in common, for example, that the pupillometric measurement data is measured in a different lighting state than the desired lighting state for which they are actually to be ascertained or acquired. The measurement is thus carried out in method steps b3) and b4) shortly after switching from the desired lighting state to a different lighting state. The pupillometric measurement data can thus be measured directly in method step b3), and in method step b4) it is calculated back to an original state by means of a model for pupil adaptation.

According to method step b3), the pupil is first adapted to the lighting state for which the pupillometric measurement data is to be ascertained and in which it cannot be measured directly in adapted form, for example. Then there is a switchover from this lighting state to another lighting state. In this other lighting state, the pupil can be measured directly, which is why this other lighting state can also be called the measurement lighting state. Measuring and acquiring the pupillometric measurement data now takes place immediately after switching from the lighting state for which the pupil is actually to be measured. The measurement is taken so soon after switching that the pupil has not yet started to adapt to the new lighting state (i.e. the measurement lighting state). This is possible because the pupil does not always start adapting immediately after a change of lighting state. The eye follows at least a constriction latency, possibly also a dilation latency. In this way, for example, the eye adapted to the brighter lighting state can be measured shortly after switching to the darker lighting state in which the eye is still de-adjusted and before the eye begins to adapt. Likewise, for example, the eye adapted to the darker lighting state can be measured shortly after switching to the brighter lighting state in which the eye is still de-adjusted and before the eye begins to adapt.

These latency periods can be several hundred milliseconds long for the transition from a bright lighting state to a darker one, for example from about 100 milliseconds to about 300 milliseconds, more precisely from about 180 milliseconds to about 230 milliseconds. Therefore, the pupillometric measurement data for the desired lighting state can be measured directly within this latency period after switching, e.g. within the first approximately 200 milliseconds after switching out of the desired lighting state. The pupil is thereby de-adjusted with regard to the measurement lighting state, as it still has the shape with which it is adapted to the desired lighting state. Thus, according to method step b3), a possible variant for determining the pupillometric measurement data of the desired lighting state is provided.

This very fast acquisition of the pupillometric measurement data according to method step b3) is not always technically feasible. Therefore, the pupillometric measurement data of the target lighting state can also be determined e.g. according to method step b4).

According to method step b4), a measurement of the pupillometric measurement data for the desired lighting state also takes place relatively promptly after a switchover from the desired lighting state to another lighting state deviating therefrom, for example to the measurement lighting state. Here, however, it is no longer the completely de-adjusted pupil that is measured, which is still adapted to the past lighting state, but the currently adapting pupil. With the help of a model, additional assumptions and a measurement of the time period from switching between the lighting states at the time of measurement, it is possible to infer the pupillometric measurement data in the original, desired lighting state. This is described in more detail below with reference to FIG. 2. In method step b4), the pupillometric measurement data is thus calculated back to the original state of the pupil in the originally set lighting state.

According to method step b2), the pupillometric measurement data is measured in exactly the lighting state for which it is also to be ascertained. The measurement is taken before the pupil has fully adjusted and/or adapted to this lighting state.

In the context of the invention, the terms adapt and adjust may be used as synonymous.

Thus, in method step b2), a switch is made from another lighting state to the lighting state in which the pupil is measured directly. However, since adaptation, in particular to a darker lighting state, can take a relatively long time, e.g. about 20 minutes, in some cases even one to two hours (see also FIG. 3 described below), the measurement is carried out well before complete adaptation and the pupillometric measurement data is extrapolated to the fully adapted target state of the pupil in the desired lighting state.

In this method step b2) the pupil is measured while it is still changing in shape. The shape that is currently changing is measured and projections are made to the fully adapted shape of the pupil in the desired lighting state. These projections can be made on the basis of a model of pupil movement.

In these exemplary embodiments, the pupillometric measurement data can either be acquired or ascertained with a very high degree of accuracy, for example by means of method steps b1) and/or b3), or with sufficient accuracy despite technical obstacles in the acquisition or ascertainment according to method step b2) and/or b4.

In a further development of this embodiment, in step b3) the measurement of the pupillometric measurement data is performed with the pupil de-adjusted within at most about 230 milliseconds after switching to the darker of the two lighting states. The measurement is therefore performed at most about 230 milliseconds after switching from the brighter target lighting state, preferably at most about 180 milliseconds after switching from the brighter target lighting state, particularly preferably already after a maximum of about 150 milliseconds. Since eyes normally begin to adjust with a latency of about 180 milliseconds to about 230 milliseconds after the transition to the darker measurement state in common lighting states, these approaches are particularly well suited for obtaining reliable pupillometric measurement data of the eye for the target lighting state.

In another further development of this embodiment, the conversion of the pupillometric measurement data in step b2) and/or b4) is performed by a model-based scaling under estimation and/or knowledge:
a percentage of a pupil size reached at the time of measurement; and/or
a percentage of a pupil stroke reached at the time of measurement; and/or
a latency from the switchover to the onset of a pupil response and a speed of the pupil response.

If the percentage of pupil size reached at the measurement time is deemed to be reasonable, the pupillometric measurement data in the target lighting state can also be calculated using the pupillometric measurement data measured at this measurement time and the pupillometric measurement data measured in the measurement lighting state. The same applies to the pupil stroke, i.e. the change in pupil size that has already taken place. In particular, the latency period and speed of the pupil response can be taken into account. These can be measured empirically and/or estimated based on models. Details of this are explained below in the context of exemplary embodiments. When using the pupil stroke to determine the desired pupillometric measurement data, pupillometric measurement data of the other lighting state can be ascertained first and/or additionally. These can be used to calculate the pupil stroke.

According to one embodiment, the brightness of the first and/or second lighting state is acquired. This can be done, for example, by means of a brightness sensor. The brightnesses acquired in this way can, for example, be acquired in lux or a similar unit of measurement and incorporated into the optical parameters. The acquired brightness values can be taken into account when creating the highly optimized and/or individual eyeglass lenses.

According to one embodiment, the method step a), the method step b), the method step c), and/or d) are repeated for a different viewing distance of the at least one eye. In particular, the objective measurement data ascertained or acquired in steps b), c) and d) can be repeated for the other viewing distance. The viewing distance can be the distance to a target object used in the subjective refraction in method step a). Thus, method steps a), b) and c) or d) can be carried out once for a near reference point and once for a far distance point. This generates additional optical measurement data that can be taken into account when creating highly optimized and/or individual eyeglass lenses and can improve their quality. The method steps a), b) and c) or d) can first be carried out for a far reference point and then at least the method steps b) and c) or d) can be repeated for a near reference point. This is preferably done with exactly the same two lighting states. This increases the comparability of the measurement data and can improve the optimization of the eyeglass lenses.

According to a specific embodiment, the subjective refraction data, the pupillometric measurement data, and the aberrometric measurement data of the at least one eye is acquired, i.e. measured immediately and directly, and/or ascertained in the first lighting state. The pupillometric measurement data of the at least one eye in the second lighting state is either directly measured and thereby acquired or is ascertained from data measured during the adjustment of the pupil to the first lighting state and thereby provided. The aberrometric measurement data of the at least one eye in the second lighting state is calculated from the acquired aberrometric measurement data in the first lighting state taking into account the acquired and/or ascertained pupillometric measurement data of the at least one eye. This is a particular embodiment according to a preferred exemplary embodiment. In this case, both the pupillometric measurement data and the aberrometric measurement data are acquired and/or ascertained in exactly the same, namely the first, lighting state as well as the subjective refraction data. It can be assumed here that, at least when acquiring the subjective refraction data, an observation beam path through a refraction unit is open and thus accessible also for the measurement of the pupillometric and aberrometric measurement data. The pupillometric measurement data for the second lighting state can be ascertained or acquired in particular by means of one of the method steps b1), b2), b3 and/or b4) described above. In this context, method steps b2) to b4) may be particularly appropriate if the observation beam path is blocked, restricted and/or reduced in the second lighting state. Finally, the aberrometric measurement data for the second lighting state can be ascertained from the aberrometric measurement data for the first lighting state, which have already been acquired, and the pupillometric measurement data. This provides sufficient optical measurement data and parameters to design and manufacture highly optimized eyeglass lenses.

According to one embodiment, the aberrometric measurement data of the at least one eye is acquired in the first lighting state before the subjective refraction data is acquired. This previously recorded aberrometric measurement data is used as the starting point of the subjective refraction. Since it can be assumed that the result of the subjective refraction at least approximately corresponds to the aberrometric measurement data for the same first lighting condition, the subjective refraction can be shortened, accelerated and/or improved with this approach.

According to one embodiment, at least the following acquired and/or ascertained data is used, as universal refraction data, to create at least one individual eyeglass lens for the user:
the subjective refraction data in the first lighting state; and/or
the pupillometric measurement data in the first and second lighting states; and/or
the aberrometric measurement data in the first and second lighting states.

In addition to this, the above measurement data can be ascertained for both a near reference point and a far reference point. Taking into account the objective measurement data, i.e. at least the aberrometric measurement data and, if applicable, also the pupillometric measurement data, can be used to adapt the individual eyeglass lens significantly better to the at least one eye of the user than is possible by merely taking into account the subjective refraction data. This applies in particular to the creation of ophthalmic eyeglass lenses. Here, data for the creation of at least one individual eyeglass lens can be processed and, for example, digitally transmitted to a manufacturer for the production of the eyeglass lens.

One aspect relates to a measuring device for determining objective measurement data of at least one eye of a user during a subjective refraction, comprising:
a refraction unit for acquiring subjective refraction data of the at least one eye in a first lighting state;
a pupil measuring unit for acquiring and/or ascertaining pupillometric measurement data of the at least one eye in the first and a second lighting state different from the first lighting state;
an aberrometry measuring unit for acquiring aberrometric measurement data of the at least one eye in at least one of the two lighting states; and
an aberrometry ascertaining unit for ascertaining aberrometric measurement data of the at least one eye in the other of the two lighting states taking into account the acquired and/or ascertained pupillometric measurement data of the at least one eye.

The measuring device can be used to perform the method according to the aspect described above. Therefore, the explanations on the method also relate to the measuring device and vice versa.

The measuring device is preferably designed as a single device in which the refraction unit, the pupil measuring unit, the aberrometry measuring unit and, if necessary, also the aberrometry ascertaining unit are integrated. To perform the method, a display unit may further be used, e.g. at least one vision panel or display, to provide and/or perform the sight exercises for subjective refraction. This display unit may be separate from the measuring device and may interact with the measuring device. For example, an electronic display such as a vision panel may receive control signals from the measuring device. The measuring device can be designed as a monocular and/or binocular measuring device.

The aberrometry ascertaining unit can be designed to ascertain the aberrometric measurement data for a target lighting state according to method steps d1), d2) and/or d3) described above from a measurement lighting state.

The measuring device may further have a control unit and/or be connected to a control unit, which may have a processor, for example. The control unit can be configured and/or used for evaluating and/or converting and/or processing the determined measurement data. For example, the control unit can be configured for image evaluation of image data acquired by the pupil measuring unit. The control unit can interact with the aberrometry ascertaining unit. The control unit can generally contribute to and/or be configured for the acquisition, evaluation, conversion and/or processing of directly measured data.

In a further development, the measuring device has a manipulation device for switching between the two lighting states by a manipulation of an observation beam path through the refraction unit, wherein the manipulation device comprises in particular:
an aperture in the observation beam path through the refraction unit, and/or
a light source, and/or
a beam path interruption at the observation beam path through the refraction unit; and/or
a filter at the observation beam path through the measuring device.

The manipulation device of the measuring device enables switching between the at least two lighting states independently of the actual lighting conditions in the measuring room in which the measurement is carried out with the measuring device.

One aspect relates to a computer program product comprising computer-readable program parts which, when loaded and executed, cause a measuring device according to claim 15 or 16 to perform a method according to any one of claims 1 to 14, wherein the computer program product at least partially controls and/or regulates at least one of the following units:
the refraction unit;
the pupil measuring unit,
the aberrometry measuring unit;
the aberrometry ascertaining unit;
a manipulation device for switching between the two lighting states; and/or
an eyeglass lens data creation unit for creating and/or calculating at least one individual eyeglass lens from the acquired measurement data.

Using the computer program product, the measurement of the refraction data, the pupillometric measurement data and/or the aberrometric measurement data can at least be supported, which can be used for a semi-automatic or even a fully automatic measurement.

Here, the lens data creation unit can be adapted to create data for creating the at least one individual eyeglass lens. This data can, for example, be transmitted digitally to a manufacturing device with which the eyeglass lens can be produced.

In the context of the present invention, the terms “substantially” and/or “about” may be used to include a deviation of up to 5% from a numerical value following the term, a deviation of up to 5° from a direction following the term and/or from an angle following the term.

Terms such as upper, lower, above, below, lateral, etc. refer—unless otherwise specified—to the earth's reference system in an operating position of the subject matter of the invention.

### BRIEF DESCRIPTION OF THE DRAWINGS

The invention is described in more detail below with reference to exemplary embodiments shown in the figures and to some embodiments independent of the figures. In this context, identical or similar reference numerals may identify identical or similar features of the embodiments. Individual features shown in the figures, for example, may be implemented in other exemplary embodiments. In the figures:

FIG. 1 shows a schematic representation of a beam path through a refraction unit of a measuring device according to an embodiment;

FIG. 2 shows a schematic, graphic representation of the dependence of a pupil reaction on different lighting states according to a first model; and

FIG. 3 shows a schematic, graphic representation of the dependence of a pupil reaction on different lighting states according to a second model.

### DETAILED DESCRIPTION

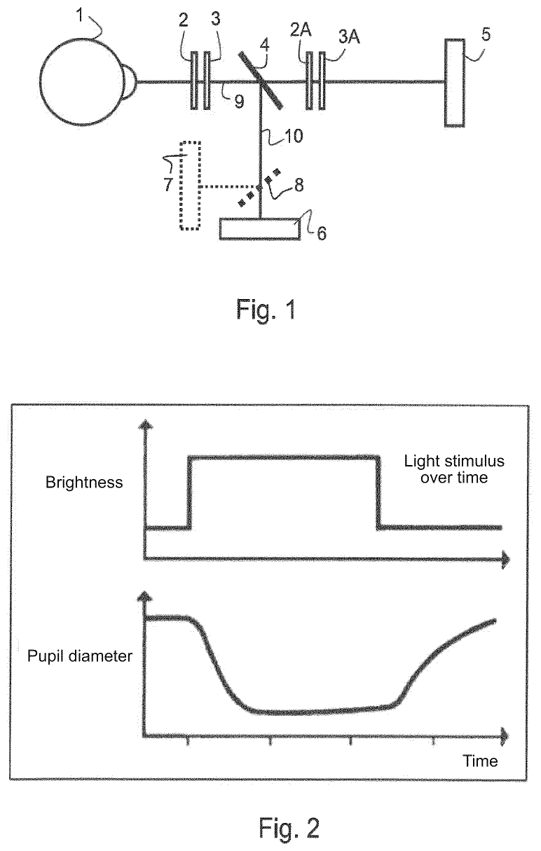

FIG. 1 shows a schematic representation of a beam path 9 through a refraction unit of a measuring device according to an embodiment. The beam path 9 thereby at least partially overlaps an observation beam path and extends from an eye 1 of a user (not shown) through the refraction unit to a display 5. The observation beam path of the refraction unit can be formed as the part of this beam path 9 that runs through the interior of the refraction unit not specifically marked in FIG. 1.

The refraction unit be used for acquiring subjective refraction data, in particular as part of a subjective refraction. In subjective refraction, the user may, for example, be shown visual symbols and/or other visual objects on the display 5, which the user is to recognize as part of a sight exercise. Subjective refraction data is thus acquired, e.g. which optical correction leads to a subjectively good visual result for the user.

In the beam path 9, starting from the eye 1, an aperture 2, an optical correction 3 and a beam splitter 4 are arranged before the user looks at the display 5. Alternatively, in the beam path 9, starting from the eye 1, first the beam splitter 4 and then an aperture 2A and an optical correction 3A can be arranged before the user looks at the display 5.

The aperture 2 or 2A can be installed in a magazine with other elements, e.g. with at least one polarizer, red filter, green filter, and/or grey filter.

The optical correction 3 or 3A can be designed as at least one phoropter lens. At this position, a plurality of phoropter lenses can be arranged one behind the other as optical corrections in the beam path 9, e.g. a lens for spherical correction and/or a lens for cylindrical correction. The optical correction 3 or 3A can be provided by a phoropter as a refraction unit. The phoropter may have a plurality of phoropter lenses, one or more of which may be selected and introduced into the beam path 9 as an optical correction 3 or 3A. A phoropter lens may be designed as a variable-strength lens.

The positions of the optical correction 3 or 3A and the aperture 2 or 2A can be interchanged. Likewise, one of these elements can be placed between the eye 1 and the beam splitter 4 and the other between the beam splitter and the display 5. The optical correction 3 or 3A and the aperture 2 or 2A can be arranged in a magazine of the refraction unit, from which elements can be introduced into the beam path 9 and the observation beam path.

The display 5 can be designed as a visual symbol panel and/or a display.

The beam splitter 4 can be configured to couple a measuring beam path 10 into the beam path 9. The measuring beam path 10 can lead to an aberrometry measuring unit 6, which can be designed as a wavefront sensor, e.g. a Shack-Hartmann sensor. The aberrometry measuring unit 6 can have an optical system (not shown in more detail in FIG. 1). The aberrometry measuring unit 6 is configured to measure and/or acquire aberrometric measurement data of the eye 1.

In one embodiment, the aberrometry measuring unit 6 can additionally measure and thereby acquire pupillometric measurement data, wherein it can be used as a pupil measuring unit at the same time.

Alternatively, a measuring beam splitter 8 can be formed in the measuring beam path 10 for coupling an optical axis of a separate pupil measuring unit 7 into the measuring beam path 10. The pupil measuring unit 7 can, for example, be designed as a camera and generate and/or acquire pupillometric measurement data from the eye 1 via the measuring beam splitter 8 and the beam splitter 4. By means of the pupil measuring unit 7, for example, a pupil size can be ascertained, in particular if this cannot be ascertained sufficiently accurately by the aberrometry measuring unit 6. The pupil measuring unit 7 can also be used independently for other objects, e.g. for eye tracking.

The refraction unit (comprising the optical correction 3 and/or 3A, the aperture 2 and/or 2A), the aberrometry measuring unit 6 and the pupil measuring unit 7 can together provide the measurement device according to an aspect of the invention. The beam splitter 4 and/or the measuring beam splitter 8 can either be formed separately to these units or integrated as a component thereof. For example, the beam splitter 4 can be integrated into the refraction unit and the measuring beam splitter 8 into the aberrometry measuring unit 6 and/or the pupil measuring unit 7.

### Exemplary Embodiments for Ascertaining the Aberrometric Measurement Data for the Other Lighting State

To acquire universal refraction data, the measuring device shown in FIG. 1 can be used, for example, to additionally acquire objective measurement data of the at least one eye 1 of the user during a subjective refraction. In any case, subjective refraction data of the eye 1 is acquired in a first lighting state. This first lighting state can, for example, be designed as a bright lighting state.

Furthermore, pupillometric measurement data of the eye 1 is acquired in the first lighting state and additionally in a second lighting state. This second lighting state may be darker than the first in some embodiments. The different lighting states can be realized with the help of a manipulation device, e.g. with the aperture 2 and/or 2A shown in FIG. 1.

In one embodiment, the aberrometric measurement data of the eye 1 is also acquired in both the first and the second lighting state. As the aberrometric measurement data for both brightness conditions is measured directly, further calculation may be superfluous here. This is a preferred embodiment, as no assumptions that may influence the data need to be made in order to collect the required data.

In one embodiment, the aberrometric measurement data for the darker lighting state is measured directly and thereby acquired. The pupillometric measurement data is measured directly, at least for the brighter lighting state, and thus acquired. This makes the aberrometric data for the darker second lighting state available directly. If the pupil for the brighter lighting state is not larger than the pupil for the darker lighting state, which may occur in exceptional cases, the aberrometric data for the brighter lighting state is preferably determined by cropping it from the acquired aberrometric measurement data for the darker lighting state according to the pupil shape for this brighter lighting state; see method step d2) described above. This embodiment leads to very accurate optical parameters, as only one direct aberrometric measurement needs to be performed and in the vast majority of cases no assumptions need to be made.

Alternatively, however, method steps d1) and/or d3) are also possible. In particular, if the pupil in the brighter lighting state should be larger than the pupil for the darker lighting state in individual cases, the aberrometric data for the brighter lighting state can be ascertained as described in method steps d1) and/or d3).

In one embodiment, the aberrometric measurement data for the brighter lighting state is measured directly and the pupillometric measurement data is measured at least for the darker lighting state. This makes the aberrometric measurement data for the brighter lighting state available directly.

In the event that the pupil in the darker lighting state is larger than the pupil in the brighter lighting state, the aberrometric measurement data for the darker lighting state can be ascertained as described in method steps d1) and/or d3).

In the event that the pupil in the darker lighting state is not larger than the pupil in the brighter lighting state, the aberrometric measurement data for the darker lighting state is preferably ascertained according to method step d2). Alternatively, method steps d1) and/or d3) are also possible, but less accurate.

This last described embodiment is relevant in the event that, for example, due to technical conditions for the darker lighting state, only the pupillometric data can be acquired, but not the aberrometric data.

### Acquiring the Pupillometric Measurement Data

When switching between lighting states, care should be taken to ensure that at least subjective refraction is not performed until the pupil has adjusted to the respective new lighting condition and is thus adapted.

The response behavior of the pupil to a change between lighting states is shown schematically in FIG. 2. Here, the x-axis is not necessarily to scale.

The upper graph shows the brightness over time, while the lower graph shows the pupil diameter corresponding to exactly the same time axis as the response behavior.

At time zero, a darker (e.g. the second) lighting state prevails. Therefore, the pupil diameter is relatively large.

A short time later, the brightness is abruptly increased to a brighter (e.g. the first) lighting state, i.e. it is switched from a darker to a brighter lighting state. This causes the pupil to constrict, but not instantaneously. Instead, there is a short constriction latency, after which the pupil begins to constrict relatively quickly until it is adjusted to the brighter lighting state after some time.

Some time after this, the light switches back to the darker lighting state, which is followed by a dilatation latency of the pupil. This dilation latency can last longer than the constriction latency. After the dilatation latency, the pupil enlarges (relatively rapidly at first, then at a rather leisurely pace) until it is approximately dilated as at the beginning.

In an exemplary embodiment, it can be assumed that the pupil change follows the following phases when switching from a darker lighting state to a brighter one and back: constriction latency,
a phase of quicker constriction,
a phase of slower constriction until maximum constriction,
a phase of maximum constriction (not necessarily constant),
a phase of quicker dilation,
a phase of slower dilation until maximum dilation.

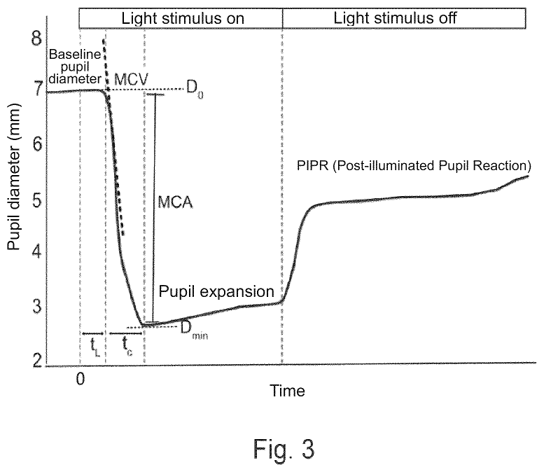

FIG. 3 shows these phases of pupil change at least schematically in a diagram. This shows the temporal change in pupil diameter as a function of a light stimulus. The diagram starts with a dark lighting state and switches to another, brighter lighting state at time 0. At a later time, it switches back to the (or another) darker lighting state. Here, too, the x-axis is not necessarily to scale.

The constriction latency t_{L} shown in FIG. 3 describes the time it takes for the pupil to react to the stronger light stimulus before it starts to contract. The constriction latency can last from about 180 ms to about 230 ms and varies with the intensity of the light stimulus and/or the ambient brightness. Usually, the stronger the light stimulus, the shorter the constriction latency.

The pupil constricts over the constriction time to from diameter D₀ to diameter Dₘᵢₙ in two phases, namely the phase of quicker constriction and the phase of slower constriction until maximum constriction. The durations of these two constriction phases may depend significantly on the absolute and/or relative intensity difference and/or on the absolute light intensity, i.e. in particular on the initial light intensity and/or the brighter light intensity. The darker the initial light intensity, the slower the respective constrictions occur and the more the maximum constriction deviates from the pupil size in the darker initial lighting state.

After the pupil has adapted to the brighter lighting state, the phase of maximum constriction follows. During this phase, the pupil size may change slightly, as shown in FIG. 3. This can cause the pupil to dilate slightly.

The speeds of dilation after a switch to the darker lighting state also depend on the stimulation intensities. The speed of dilation changes over time, wherein it is fastest immediately after the loss of the strong light stimulus, i.e. during the phase of quicker dilation, and it decreases towards the phase of slower dilation, in particular after reaching about 75% of the target amplitude. Here it can be assumed that the phase of quicker dilation takes place at a dilation speed of about 2.7 mm/s and is terminated after about 0.6 seconds. The phase of slower dilation lasts significantly longer. For example, the maximum dilation can be reached only after up to 20 minutes or even after one to two hours. The graph shown in FIG. 3 ends at the right end of the time axis in a kind of slowly rising plateau. At the end of this plateau, the pupil may already be adjusted to the darker lighting state; however, the adjustment can also take considerably longer, namely beyond the end of the time axis shown in FIG. 3. In the process, the pupil diameter may even slowly expand again up to the initial value at the left end of the time axis. This phase of slow adjustment, which may take several hours, is no longer shown in full in FIG. 3. In one embodiment, the height of the plateau shown at the right edge in FIG. 3 can be used to infer the target value of the pupil diameter in the fully adjusted state of the pupil in order to shorten the wait for a complete adjustment and/or make it superfluous.

In one embodiment, an interior brightness is used as a brighter lighting state, which is from about 300 lux to about 1000 lux. A dark room brightness with a brightness lower than about 5 lux is used as the darker lighting state. For such lighting states, which are comparatively easy to achieve, a constriction time (including constriction latency) of about 1 second is assumed overall, wherein at least about 50% of the maximum constriction is reached after about 0.4 s. The constriction speed reaches its maximum very quickly during the quicker constriction phase and changes to the slower constriction phase after about 50% of the change.

When dilating, it is expected that the first about 65% to about 75% of the maximum dilation can be achieved about 0.6 s after switching to the darker lighting state, wherein it can be assumed that the maximum dilation speed is about half the maximum constriction speed.

When using these two lighting states, the pupil needs about one second to adapt after switching from the darker to the brighter lighting state, and vice versa from the brighter to the darker lighting state about three to five seconds, in extreme cases up to 20 minutes. These empirical data and/or assumptions can be used as a model to scale e.g. the aberrations and/or pupil size in a model-based way.

This model of pupil change, which can behave as shown in FIG. 3, can be used in different ways to collect pupillometric measurement data.

In one exemplary embodiment, the dilated pupil is to be measured in a darker lighting state without waiting for the full adaptation time. This can be done, for example, as part of the previously introduced method step b2), in which the pupillometric measurement data is to be ascertained for an e.g. darker lighting state, and can be carried out, for example, as follows:

A percentage of the pupil size reached at the onset of the measurement may be known or estimated, e.g. when switching from a brighter lighting state to a darker lighting state. Here, for example, approx. 1.0 s after switching to the darker lighting state can be measured. Then it can be assumed that a transition plateau has already been reached, on which the pupil is in the phase of slower dilatation. In this measurement, however, do not wait for the maximum 20 minutes (or longer) that the pupil might take to dilate to its maximum. Here it can be assumed that at the time of measurement (i.e. approx. 1.0 s after switching) about 66% of the maximum pupil size has been reached. If a pupil size of e.g. 3 mm is measured approx. 1.0 s after switching, the size of the maximally dilated and thus adapted pupil in the darker lighting state can be estimated:

3 mm/66%=4.5 mm.

Thus, the pupil size measured during adjustment can be divided by the known or estimated percentage of the pupil size achieved at the onset of measurement to determine the pupil size at the darker target lighting value.

Alternatively or additionally, in order to carry out method step b2) in the above lighting conditions, a percentage of the pupil stroke reached at the onset of the measurement, i.e. the pupil change, can be known or estimated. For example, when switching from a brighter lighting condition to a darker lighting condition, it can be assumed that when the measurement is taken approximately 1.0 s after switching to the darker lighting condition, approximately 75% of the maximum pupil stroke has been reached. The expected pupil size in the darker lighting state can be derived from the pupil stroke. In a numerical example, a pupil size of 2 mm is initially measured in the brighter lighting state and a pupil size of 5 mm is measured 1.0 s after switching. Here, the maximum dilation has not yet been reached, the pupil is still in the phase of adaptation, in particular in the phase of slower dilation. From this, the pupil size in the darker lighting state can be derived:

2 mm+(5 mm−2 mm)/75%=2 mm+4 mm=6 mm.

Here, the pupil stroke measured during adaptation can thus be divided by the known or estimated percentage of the pupil stroke achieved at the onset of measurement and then added to the pupil size in the brighter lighting state to ascertain the pupil size at the darker target lighting value.

In one exemplary embodiment, the pupil change model, which may behave as shown in FIG. 3, may be used to measure the reduced pupil in a brighter lighting state without waiting for the full adaptation time. This can be done, for example, as part of method step b2), in which the pupillometric measurement data is to be ascertained for an e.g. brighter lighting state, and can be carried out, for example, as follows:

A percentage of the pupil size reached at the onset of the measurement may be known or estimated, e.g. when switching from a darker lighting state to a brighter lighting state. Here, approx. 0.4 s after switching to the brighter lighting state can be measured. It can then be assumed that at the time of measurement the pupil is about 150% of the minimum pupil size. If a pupil size of e.g. 3 mm is measured approx. 0.4 s after switching, the size of the minimally constricted and thus adapted pupil in the brighter lighting state can be estimated:

3 mm/150%=2 mm.

Here, too, the pupil size measured during adaptation can be divided by the known or estimated percentage of the pupil size achieved at the onset of measurement to determine the pupil size at the desired brighter lighting value.

Alternatively or additionally, in order to carry out method step b2) in the above lighting conditions, a percentage of the pupil reduction reached at the onset of the measurement, i.e. the pupil change, can be known or estimated. For example, when switching from a darker lighting condition to a brighter lighting condition, it can be assumed that when the measurement is taken approximately 0.4 s after switching to the brighter lighting condition, approximately 50% of the maximum pupil constriction has been reached. The expected pupil size in the brighter lighting state can be derived from the measured pupil constriction. In a numerical example, a pupil size of 6 mm is initially measured in the darker lighting state and a pupil size of 4 mm is measured 0.4 s after switching. Here, the pupil is not yet maximally constricted, as the pupil is still in the phase of adaptation. From this, the pupil size in the brighter lighting state can be derived:

6 mm−(6 mm−4 mm)/50%=6 mm−4 mm=2 mm.

Here, the pupil constriction measured during adaptation can thus be divided by the known or estimated percentage of the pupil stroke achieved at the onset of measurement and then subtracted from the pupil size in the darker lighting state to ascertain the pupil size at the brighter target lighting value.

In one exemplary embodiment, the dilated pupil is to be measured in a darker lighting state, as part of method step b3) introduced above. This can be caused, for example, by the fact that the measurement requires the switching on of lighting, as it is not possible to measure directly in the darker lighting state.

To carry out method step b3), the pupil can still be measured within the latency period. For example, this relates to the phase of the constriction latency when switching from a darker lighting state to a brighter lighting state. Here, for example, approx. 150 ms after switching to the brighter lighting state can be measured. At this point, the pupil still has its original size, so the measured value does not need to be corrected.

In a similar exemplary embodiment, the dilated pupil is also to be measured in a darker lighting state, as part of method step b4) introduced above. This can also be caused, for example, by the fact that the measurement requires the switching on of lighting, as it is not possible to measure directly in the darker lighting state.

This method step b4), in which the pupillometric measurement data is to be ascertained for an e.g. brighter lighting state, can be carried out, for example, as follows:

The measurement takes place after a switchover from the desired darker lighting state, for which the pupillometric measurement data is to be ascertained, to the brighter measurement lighting state, in which the pupil is actually measured. The measured data is calculated back to the original, brighter lighting state. Here, a time until the onset of the pupil reaction and a change speed, e.g. the constriction speed, may be known or estimated. For example, measurements can be taken approx. 0.5 s after switching from the brighter lighting state to the darker lighting state. Then a constriction latency of about 0.2 s and a constriction speed of about 5.5 mm/s can be assumed. Here, the maximum constriction has not yet been reached, the pupil is still adapting. Thus, after the latency period 0.5 s−0.2 s=0.3 s, during which the pupil has shrunk by 0.3 s*5.5 mm/s=1.65 mm. If, in a numerical example, a pupil size of 4.0 mm is measured 0.5 s after switching, this results in a maximum pupil size in the desired darker lighting state of:

4.0 mm+1.65 mm=5.65 mm.

The above embodiments are provided as exemplary calculations. Alternatively, more complex correction modules can be used, by means of which the calculation can be improved. Furthermore, the shape and any pupil shifts can also be taken into account.

For switching from the brighter to the darker lighting state for performing method step b4), the same procedure can be followed as for switching from the darker to the brighter lighting state described above. The pupillometric measurement data for the brighter lighting state can be calculated from the pupillometric measurement data for the darker lighting state and a model of pupil behavior.

For example, due to the construction, it may be difficult to directly measure the pupillometric measurement data for the darker lighting condition. If the darker lighting state is produced, e.g. by closing the beam path 9 by means of the aperture 2 (see FIG. 1), the aperture 2 also blocks the optical axis of the pupil measuring unit, e.g. the pupil measuring unit 7 shown in FIG. 1, which is deflected around the beam splitter 4 and possibly the measuring beam splitter 8.

This means that the pupillometric measurement data for the eye 1 can only be measured directly in the brighter lighting state with the pupil adapted. Then the darker lighting state can be established and wait at least 3-5 seconds or longer for the eye 1 to get used to it. Afterwards, the aperture 2 can be opened to create the brighter lighting state. The pupillometric measurement data is measured again, relatively soon after the switchover, e.g. about 0.4 s after the switchover. It can then be assumed that the pupil has constricted to about 50% of the maximum constriction amplitude. From the pupillometric measurement data measured in this way during the adjustment, the pupillometric measurement data in the darker lighting state can be inferred with the aid of the model in order to ascertain and/or acquire this data in this way and, for example, to carry out method step b4).

When acquiring pupillometric measurement data, the lighting state are preferably set for both eyes in order to prevent the eye being measured from being influenced by the other eye.

For design reasons, it may be that the measurement of the pupillometric and/or aberrometric measurement data can only be carried out with the observation beam path open or closed (e.g. the beam paths 9 and/or 10 shown in FIG. 1) and/or with or without lighting. This means that these measurements can only be made directly and immediately in one of the two lighting states and are blocked in the other.

A possible cause for this may be that an aperture blocking the observation beam path is blocking the measurement beam path, see aperture 2 in beam paths 9 and 10. An optical element that generates a lighting beam path, e.g. a mirror, can block this observation beam path. Additional illumination, which is used e.g. to create the brighter or defined darker lighting state, can block the measurement beam path. Alternatively or additionally, the measurement can be disturbed by stray light, for example.

Therefore, if the measurement cannot be carried out in the actually desired lighting state (e.g. the brighter or darker one), e.g. because the subjective refraction takes place in the brighter lighting state and an aperture of the observation beam path also blocks the measuring beam path, the following procedure can be followed, for example:
1. The desired lighting state, i.e. the one that is actually to be measured (e.g. the darker one), is set, e.g. by closing an aperture and/or reducing an opening of a pinhole for which the pupillometric and/or aberrometric measurement data are to be ascertained. Here, it is preferable to wait until the pupil reaction is complete, i.e. until the pupil has adapted.
2. The lighting state (e.g. the brighter one) of the measuring device necessary for measurement, i.e. the measurement lighting state, is established, e.g. by opening the shutter, since measurement is not possible with the shutter closed.
3A. The measurement of at least the pupillometric data can be performed before the onset of a measurable pupil reaction, e.g. still in the constriction latency or dilatation latency, e.g. as according to method step b3).
3B. If the measurement can only be made after the onset of a measurable pupil reaction, the measured pupillometric data can be adjusted and/or scaled to the desired, original lighting state as described above, e.g. as per method step b4). The aberrometric measurement data belonging to the desired, original lighting state can be determined by means of the already recorded pupillometric measurement data, see method steps d1), d2), and d3) described above.

The adaptation of the pupillometric data, in particular the pupil size, can be carried out in this case and/or according to method step b4), for example, as follows in the event that the constricted pupil is to be measured in the brighter lighting state as the desired lighting state, i.e. the lighting state that is actually to be measured, but a darkening (i.e. the darker lighting state) is required for the measurement:
3B1. In a first variant, the assumptions are made that there is no latency period in the dilation of the pupil and also an assumption of the dilation speed, e.g. that in the phase of quicker dilation, a quicker dilation speed is about 2.7 mm/s. Now measure in the phase of rapid expansion, e.g. about 0.3 s after switching from the brighter to the darker operating state. The pupil can therefore be dilated by 0.3 s*2.7 mm/s=0.81 mm at this point. In a numerical example, if a pupil size of 3.50 mm is measured 0.3 s after switching, the initial value of for the minimum pupil size is:

3.50 mm−0.81 mm=2.69 mm.
3B2. In a second variant of this scenario, an assumption is made about a percentage of a pupil stroke reached at a given time. For example, it can be assumed that approx. 1.0 s after switching, the dilation plateau (=phase of slower dilation) has already been reached. At this point, for example, about 75% of the maximum pupil stroke may have been reached. If a pupil size of 4.00 mm is taken into account at this point, and in addition a pupil size of 6.00 mm previously measured in the dark, this results in an expected total stroke of (6 mm−4 mm)/75%=2.67 mm. This allows the minimum pupil size in the brighter operating state to be estimated:

6.00 mm−2.67 mm=3.33 mm.
3B3. In a third variant of this scenario, measurements are taken after reaching the dilation plateau (=phase of slower dilation), e.g. also approx. 1.0 s after switching to the darker operating state. Assumptions can be made as to what percentage of the minimum exit pupil has been reached. For example, it can be assumed that at this point the pupil has already dilated to, say, about 150% of the minimum exit pupil. If a pupil size of 3.00 mm is measured at this time, the minimum pupil in the brighter operating state results in:

3.00 mm/150%=2.00 mm.

These exemplary embodiments 3B1, 3B2 and 3B2 are given as numerical examples for illustrative purposes. The calculations only serve to explain how the desired pupillometric measurement data can be determined for the desired lighting state.

In the opposite case, i.e. when determining the pupillometric measurement data for the darker lighting state, the procedure can be analogous.

More complex mathematical models of pupil movement can also be considered, such as a sigmoid progression and/or a progression according to the equation:

d(t)=dₘᵢₙ+Δd*(1−exp(−(t−t_{L})/τ)).

Here d(t) describes the diameter and thus the size of the pupil at time t, dₘᵢₙ the diameter of the minimum pupil in the brighter lighting state, Δd the pupil stroke, t the time, t_{L} the constriction latency, and τ a time constant. The parameters of this equation can be at least partially predefined, e.g. empirically ascertained, and/or at least partially adapted to the measurement data.

### Acquiring the Objective Refraction Data at Different Distances

The method described above can basically be used for all viewing distances. Preferably, however, it is carried out for the far distance, i.e. with a distance of preferably at least 5 m.

In the method, the aberrometric measurement data is generated for two different conditions, namely for the two different lighting states. However, the data does not necessarily have to differ in terms of brightness. Rather, the measurement conditions may additionally or instead differ based on the viewing distance used in the measurement.

Preferably, a far distance measurement is combined with a near measurement. Near measurements are taken, for example, at 40 cm as the usual close-up distance, or from about 20 cm to about 30 cm for working with hand-held devices such as mobile phones or tablets.

To create space and/or near comfort lenses, a corresponding space and/or working distance (of e.g. 3 m) can be used instead of the refraction at a distance.

The pupil size and/or shape and/or position and/or addition can be detected for near distance. Preferably, a more extensive data set than aberrometric measurement data is acquired, i.e. including e.g. an axis, a cylinder and/or a higher order aberration.

If a display with visual objects as one target is viewed binocularly by a phoropter, a slow approach to the target can be dispensed with, as the user's visual system then has information about the binocular disparity.

### Combination of States at Different Brightnesses With States at Different Distances

Preferably, these two differentiating conditions, i.e. brightness and distance, are combined.

In a preferred embodiment, the method is carried out for two different brightnesses for the far distance and additionally at least one measurement is carried out for the near distance.

### First Exemplary Embodiment

A subjective refraction for the distance for a brighter first lighting state is performed comprising the following steps:
1. binocularly acquiring the pupillometric and aberrometric measurement data for the brighter first lighting state;
2. subjective refraction for the far distance for the brighter first lighting state individually for both eyes of the user, with a binocular adjustment if necessary, and using the already acquired aberrometric measurement data as a starting point if necessary;
3. setting the darker second lighting state and, after sufficient waiting time, binocularly acquiring pupillometric measurement data for the darker second lighting state; wherein
   the darker second lighting state can be produced by at least one aperture in the observation beam path;
   pupillometric measurement data can be measured;
   if necessary, a correction of the measured pupillometric data is performed to obtain the corrected pupillometric data for the darker second lighting condition;
   the aberrometric measurement data for the darker second lighting state is determined from the already acquired aberrometric measurement data for the brighter first lighting state and the pupillometric measurement data; or
   alternatively: the aberrometric measurement data for the darker second lighting state is measured directly, wherein a disadvantageous device myopia may be present due to the darkening, which is why, if necessary, a correction of the aberrometric measurement data can be carried out as described above;
4. subjective refraction for the near distance for the brighter first lighting state individually for both eyes of the user, with a binocular adjustment if necessary; and
5. measuring the pupillometric and aberrometric measurement data for the brighter first lighting state.

Steps 4 and 5 are optional, but improve the overall quality of the measurement data.

### Second Exemplary Embodiment

A subjective refraction for the distance for a darker first lighting state is performed comprising the following steps:
1. binocularly acquiring the pupillometric and aberrometric measurement data for the darker first lighting state;
2. subjective refraction for the far distance for the darker first lighting state individually for both eyes of the user, with a binocular adjustment if necessary, and using the already acquired data, if necessary
3. setting the brighter second lighting state and, after sufficient waiting time, binocularly acquiring pupillometric measurement data for the brighter second lighting state; wherein
   the brighter second lighting state can be produced by illumination;
   pupillometric measurement data can be measured;
   if necessary, a correction of the measured pupillometric data is performed to obtain the corrected pupillometric data for the brighter second lighting condition;
   the aberrometric measurement data for the brighter second lighting state is determined from the already acquired aberrometric measurement data for the darker first lighting state and the pupillometric measurement data; or
   alternatively: the aberrometric measurement data for the brighter second lighting state is measured directly, wherein a disadvantageous device myopia may be present due to the darkening, which is why, if necessary, a correction of the aberrometric measurement data can be carried out as described above;
4. subjective refraction for the near distance for the darker first lighting state individually for both eyes of the user, with a binocular adjustment if necessary; and
5. measuring the pupillometric and aberrometric measurement data for the darker first lighting state.

Here, too, steps 4 and 5 are optional and only of limited use for improving the quality of the measurement data.

The above exemplary embodiments may be performed in a different order as long as at least the following are acquired:
aberrometric measurement data for a lighting state;
pupillometric measurement data for both lighting states; and
subjective refraction data for a lighting state.

In this case, the lighting state in which the subjective refraction is performed may differ from the lighting states in which the pupillometric and/or aberrometric measurement data are acquired.

### LIST OF REFERENCE NUMERALS

1 Eye
2 Aperture
2A Aperture
3 Optical correction
3A Optical correction
4 Beam splitter
5 Display
6 Aberrometry measuring unit
7 Pupil measuring unit
8 Measuring beam splitter
9 Beam path
10 Measuring beam path

1-17. (canceled)

18. A method for determining objective measurement data of at least one eye of a user during a subjective refraction, the method comprising:
a) acquiring subjective refraction data of the at least one eye in a first lighting state;
b) acquiring and/or ascertaining pupillometric measurement data of the at least one eye in the first and a second lighting state different from the first lighting state; and
c) acquiring aberrometric measurement data of the at least one eye in the first and second lighting states;
or
d) acquiring aberrometric measurement data of the at least one eye in the first or the second lighting state and ascertaining aberrometric measurement data of the at least one eye in the other one of the first and second lighting states taking into account the acquired and/or ascertained pupillometric measurement data of the at least one eye.

19. The method according to claim 18, wherein the method is carried out using a single measuring device into which a refraction unit configured to acquire the subjective refraction data, a pupil measuring unit configured to acquire the pupillometric measurement data, and an aberrometry measuring unit configured to acquire the aberrometric measurement data are integrated.

20. The method according to claim 18, further comprising:
switching between the lighting states without changing an ambient lighting condition.

21. The method according to claim 18, wherein a switch between the lighting states is effected by changing a brightness of a display unit;
and/or
wherein the subjective refraction is carried out along an observation beam path and wherein a switch between the lighting states is effected by a manipulation of the observation beam path, wherein the manipulation is effected by:
changing an aperture; and/or
operating a light source; and/or
operating a beam path interruption; and/or
operating a filter.

22. The method according to claim 18, wherein in step d), the aberrometric measurement data of the at least one eye in the other of the first and second lighting states are ascertained by:
d1) representing the already acquired aberrometric measurement data in the first or second lighting state in a set of coefficients, in particular in Zernike coefficients, and scaling this set of coefficients using the acquired and/or ascertained pupillometric measurement data to the other of the first and second lighting states, and/or
d2) cutting out aberrometric data of a pupil shape according to the pupillometric measurement data acquired and/or ascertained for the other lighting state from the acquired aberrometric measurement data of a pupil shape according to the acquired and/or ascertained pupillometric measurement data belonging to the lighting state for which also the aberrometric measurement data were acquired, and/or
d3) extrapolating the already acquired aberrometric measurement data in the first or second lighting state to the pupil shape according to the acquired and/or ascertained pupillometric measurement data in the other lighting state.

23. The method according to claim 18, wherein in step b):
b1) the pupillometric measurement data of the at least one eye in at least one of the first and second lighting states are directly measured using a pupillometric measurement of the pupil of the at least one eye fully adapted to this lighting state performed during this lighting state; and/or
b2) the pupillometric measurement data of the at least one eye are indirectly ascertained in at least one of the first and second lighting states using a pupillometric measurement performed during this lighting state of the pupil of the at least one eye which is currently adapting to this lighting state, namely
after a switchover from another lighting state to this lighting state; and
before the pupil has fully adapted to this lighting state; and
using a conversion of this pupillometric measurement data measured during the adaptation to an adapted target state of the pupil in this lighting state.

24. The method according to claim 23, wherein in step b):
b3) the pupillometric measurement data of the at least one eye are directly measured in at least one of the first and second lighting states using a pupillometric measurement of the pupil of the at least one eye still fully adapted to this one of the first and second lighting states performed during another lighting state, and immediately after a switchover from this lighting state to the other lighting state and before the pupil starts adapting to the other lighting state; and/or
b4) the pupillometric measurement data of the at least one eye is indirectly ascertained in at least one of the first and second lighting states using a pupillometric measurement performed during another lighting state of the pupil of the at least one eye which is currently adapting to this other lighting state, namely
after a switchover from the lighting state for which the pupillometric measurement data are to be ascertained to the other lighting state; and
while the pupil is still adapting to this other lighting state; and
using a conversion of this pupillometric measurement data measured during the adaptation to an adapted initial state of the pupil in this original lighting state.

25. The method according to claim 24, wherein in step b3), the measurement is performed within at most 230 ms after the switchover to a darker one of the first and second lighting states.

26. The method according to claim 24, wherein in step b2) and/or step b4), the conversion of the pupillometric measurement data is performed by a model-based scaling with estimation and/or knowledge of:
a percentage of a pupil size reached at a time of measurement; and/or
a percentage of a pupil stroke reached at the time of measurement; and/or
a latency from the switchover to an onset of a pupil response and a speed of the pupil response.

27. The method according to claim 18, further comprising:
acquiring a brightness of the first and/or second lighting state.

28. The method according to claim 18, wherein the method step a), the method step b), the method step c), and/or the method step d) are repeated for a different viewing distance of the at least one eye.

29. The method according to claim 18, wherein:
the subjective refraction data, the pupillometric measurement data, and the aberrometric measurement data of the at least one eye is acquired and/or ascertained in the first lighting state;
the pupillometric measurement data of the at least one eye in the second lighting state is either directly measured and thereby acquired or is ascertained from data measured during an adjustment of the pupil to the first lighting state; and
the aberrometric measurement data of the at least one eye in the second lighting state is calculated from the acquired aberrometric measurement data in the first lighting state taking into account the acquired and/or ascertained pupillometric measurement data of the at least one eye.

30. The method according to claim 18, wherein the aberrometric measurement data of the at least one eye in the first lighting state are acquired before the subjective refraction data are acquired, and wherein this previously acquired aberrometric measurement data is used as a starting point of the subjective refraction.

31. The method according to claim 18, wherein at least the following acquired and/or ascertained data is used, as universal refraction data, to create at least one individual eyeglass lens for the user:
the subjective refraction data in the first lighting state; and/or
the pupillometric measurement data in the first and second lighting states; and/or
the aberrometric measurement data in the first and second lighting states.

32. A measuring device for determining objective measurement data of at least one eye of a user during a subjective refraction, comprising:
a refraction unit configured to acquire subjective refraction data of the at least one eye in a first lighting state;
a pupil measuring unit configure to acquire and/or ascertain pupillometric measurement data of the at least one eye in the first and a second lighting state different from the first lighting state;
an aberrometry measuring unit configured to acquire aberrometric measurement data of the at least one eye in at least one of the first and second lighting states; and
an aberrometry ascertaining unit configured to ascertain aberrometric measurement data of the at least one eye in the other of the first and second lighting states taking into account the acquired and/or ascertained pupillometric measurement data of the at least one eye.

33. The measuring device according to claim 32, comprising a manipulation device configured to switch between the first and second lighting states by a manipulation of an observation beam path through the refraction unit, wherein the manipulation device comprises:
an aperture in the observation beam path through the refraction unit; and/or
a light source; and/or
a beam path interruption at the observation beam path through the refraction unit; and/or
a filter at the observation beam path through the measuring device.

34. A non-transitory computer program product comprising computer-readable program parts which, when loaded and executed, cause a measuring device for determining objective measurement data of at least one eye of a user during a subjective refraction, including a refraction unit configured to acquire subjective refraction data of the at least one eye in a first lighting state, a pupil measuring unit configure to acquire and/or ascertain pupillometric measurement data of the at least one eye in the first and a second lighting state different from the first lighting state, an aberrometry measuring unit configured to acquire aberrometric measurement data of the at least one eye in at least one of the first and second lighting states, and an aberrometry ascertaining unit configured to ascertain aberrometric measurement data of the at least one eye in the other of the first and second lighting states taking into account the acquired and/or ascertained pupillometric measurement data of the at least one eye, to perform a method according to claim 18, wherein the non-transitory computer program product at least partially controls and/or regulates a unit selected from a group of units comprising:
the refraction unit;
the pupil measuring unit;
the aberrometry measuring unit;
the aberrometry ascertaining unit;
a manipulation device configured to switch between the first and second lighting states; and/or
an eyeglass lens data creation unit configured to create and/or calculate at least one individual eyeglass lens from the acquired measurement data.
